(19) United States
(12) Patent Application Publication
(10) Pub. No.: US 2022/0088130 A1
(43) Pub. Date: Mar. 24, 2022

(54) COMPOUNDS AND METHODS FOR TREATING LIVER DISEASES

(71) Applicant: Eberhard Karls Universität Tübingen Medizinische Fakultät, Tuebingen (DE)

(72) Inventors: Lusine DANIELYAN, Tuebingen (DE); Gayane BUNIATIAN, Tuebingen (DE); Matthias SCHWAB, Stuttgart (DE); Ralf WEISKIRCHEN, Aachen (DE); Thomas WEISS, Regensburg (DE); Christoph Hermann GLEITER, Tuebingen (DE)

(21) Appl. No.: 17/449,723

(22) Filed: Oct. 1, 2021

Related U.S. Application Data
(63) Continuation of application No. PCT/EP2020/059486, filed on Apr. 3, 2020.

(30) Foreign Application Priority Data
Apr. 4, 2019 (DE) 102019108825.9

(51) Int. Cl. A61K 38/17; A61K 38/48; A61K 31/216; A61K 35/30; A61K 35/28; A61P 1/16

(52) U.S. Cl. CPC A61K 38/1716; A61K 38/488; A61P 1/16; A61K 35/30; A61K 35/28; A61K 31/216

(57) ABSTRACT

The present invention relates to a compound for use in the treatment or prevention of a liver disease, wherein the compound is a amyloid beta related protein, the amyloid beta related protein being selected from the group consisting of amyloid beta protein, a amyloid beta peptide derived therefrom, amyloid precursor protein (APP), a compound involved in the generation of an amyloid beta peptide from APP, or a compound inhibiting the degradation of the amyloid beta protein or of amyloid peptides derived therefrom.

### CROSSREFERENCES TO RELATED APPLICATIONS

This application is a continuation of international patent application PCT/EP2020/059486, filed on Apr. 3, 2020, designating the U.S., which international patent application has been published in English language and claims priority from German patent application DE 10 2019 108 825.9, filed on Apr. 4, 2019. The entire contents of these priority applications are incorporated herein by reference.

### BACKGROUND

The present invention relates to a compound for use in the treatment of liver diseases as well as to methods for treating liver diseases by means of a compound, more particularly treating liver fibrosis or cirrhosis.

The liver is an organ that performs various functions. The liver plays a vital role in synthesis of proteins (for example, albumin, clotting factors and complement), detoxification, and storage (for example, vitamin A). In addition, it participates in the metabolism of lipids and carbohydrates. In recent years, the development of liver diseases and the number of deaths caused by liver diseases have gradually increased. Liver cirrhosis has many possible causes; sometimes more than one cause is present in the same person. Globally, 57% of cirrhosis is attributable to either hepatitis B (30%) or hepatitis C (27%). Alcohol consumption is another major cause, accounting for about 20% of the cases.

Liver fibrosis/cirrhosis is a condition in which the liver does not function properly due to long-term damage. This damage is characterized by the replacement of normal liver tissue by scar tissue. Typically, the disease develops slowly over months or years.

Generally, liver cirrhosis refers to the end stage of liver disease. The major causes of liver cirrhosis are diverse, including hepatitis, viral infections, alcohol intoxication, bile acid secretion disorder, drug addiction, allergy, and excessive iron deposition.

Liver cells can be repaired by regeneration owing to their strong regenerating ability even when they are destroyed to some degree. However, after a certain point of time after liver cell destruction, the destroyed cells are not regenerated but undergo fibrosis, which results in liver hardening. This condition, in which the liver is hardened by a change in its structure so that it cannot go back to the original state, is referred to as liver cirrhosis. Therefore, liver damage from cirrhosis cannot be reversed, but treatment can stop or delay further progression and reduce complications.

Liver fibrosis refers to a disease in which liver tissue in a chronic inflammatory state is repeatedly damaged and repaired so that connective tissues such as collagen are excessively deposited in the liver tissue, thereby causing scars in the liver tissue.

Generally, unlike liver cirrhosis, liver fibrosis is reversible and is composed of thin fibrils without nodule formation. Once the cause of hepatic injury is eliminated, the liver can be returned to the normal state. However, if the liver fibrosis mechanism is continuously repeated, the liver fibrosis leads to irreversible liver cirrhosis in which crosslinking between connective tissues increases to accumulate thick fibrils, and a liver lobule loses its normal structure to cause nodule formation.

Liver diseases are caused by various causes, but if these liver diseases become chronic, they commonly lead to liver fibrosis or liver cirrhosis regardless of the causes thereof. Liver diseases are asymptomatic in the initial stage, and thus are difficult to diagnose early. Furthermore, because liver diseases are generally found in the chronic stage, these liver diseases are not easy to treat and have a high mortality rate, and thus pose social problems. In addition, therapeutic agents having excellent effects have not yet been developed.

Current drug- and cell-based therapies of liver fibrosis/cirrhosis which aim at reduction or even reversal of hepatic fibrogenesis irrespective of its etiology (primary biliary cirrhosis, nonalcoholic steatohepatitis, alcohol hepatitis, hepatocellular carcinoma and viral hepatitis) give hope for future treatments of liver fibrosis/cirrhosis by targeting the activation of hepatic stellate cells (HSC) which is the most prominent hallmark of the liver cirrhosis/fibrosis.

However, none of the candidates (either drugs or cells) are capable of showing complex effects influencing both, deactivation of HSC and the reversal of liver endothelia sinusoidal cells (LSEC) impermeability. The permeability of blood-tissue interface plays a crucial role in the maintenance of the main liver function of toxic compounds detoxification delivered by the blood.

There is currently no treatment of the progression of development of liver fibrosis or cirrhosis other than antiviral therapy, which prevents underlying hepatic destruction.

### SUMMARY

Against this background, the object of the present invention is to overcome the aforesaid deficiencies in the prior art.

It is another object of the present invention to treat liver diseases, especially liver fibrosis or cirrhosis.

It is a further object of the present invention to prevent liver diseases, especially liver fibrosis or cirrhosis.

According to the invention this object is achieved by a compound for use in the treatment or prevention of a liver disease, wherein the compound is a amyloid beta related protein which is selected from group consisting of the amyloid beta protein, a amyloid beta (Aβ) peptide derived therefrom, amyloid precursor protein (APP), a compound involved in the generation of an amyloid beta peptide from APP, or a compound inhibiting the degradation of the amyloid beta protein or of amyloid peptides derived therefrom.

The invention further relates to a eukaryotic cell that is naturally programmed, i.e. without genetic modification, to produce high levels of APP and to degrade Aβ to a less extent than hepatic stellate cells (HSC), for use in the treatment or prevention of a liver disease.

According to a preferred embodiment, the eukaryotic cell is of perivascular origin, and, thus, is able to replace activated hepatic stellate cell (HSC) in the disease liver, i.e. a fibrotic/cirrhotic liver. Hereby, the less potency of the eukaryotic cell according to the invention to degrade Aβ than compared to HSC, and the enhanced expression of APP in response to TGF-β and TNF-α, which are highly upregulated in liver diseases such as liver cirrhosis, will increase its content in the perivascular space in the liver and will, thus, contribute to deactivation of the fibrotic phenotype of hepatic stellate cells, as well as will increase the permeability of liver endothelial sinusoidal cells.

According to a preferred embodiment, the eukaryotic cell is/are selected from at least one of the following: astrocytes, iPS- (induced pluripotent stem cell)-derived astrocytes D: 30409508), and somatic cells directly reprogrammed to astrocytes (PMID: 22308465).

With a eukaryotic cell according to the invention, in particular astrocytes, liver diseases can be efficiently treated. In this connection, the inventors of the present invention have shown that an astroglial primary culture degrades less potently Aβ than hepatic stellate cells. Also, as mentioned above, due to the fact that astrocytes increase the levels of BACE1, APP, and β-secretase processing, and that astrocytic Aβ production increases in response to pro-inflammatory cytokines, such as TNF-α and TGF-β, which are upregulated during liver cirrhosis, astrocytes will increase their production of APP and—as a consequence—of Aβ, once transplanted into the cirrhotic liver.

The invention further relates to a genetically modified eukaryotic cell for use in the treatment or prevention of a liver disease, wherein the genetically modified eukaryotic cell has been modified to overexpress amyloid beta protein and/or amyloid beta peptides derived therefrom, APP, BACE1, and/or presenilin.

According to a preferred embodiment, the a genetically modified eukaryotic cell for use according to the invention is selected from at least one of the following: genetically modified mesenchymal stromal cell, genetically modified astrocytes, genetically modified iPS- (induced pluripotent stem cell)-derived astrocytes, and genetically modified somatic cells directly reprogrammed to astrocytes.

The invention further relates to a pharmaceutical composition for use in the treatment or prevention of a liver disease, especially liver fibrosis or cirrhosis, the pharmaceutical composition comprising an amyloid beta related protein, the amyloid beta related protein being selected from amyloid beta protein, an amyloid beta peptide derived therefrom, amyloid precursor protein (APP), an enzyme involved in the generation of an amyloid beta peptide from APP, or an inhibitor of the degradation of amyloid beta protein or of amyloid beta peptides derived therefrom, and/or comprising a genetically modified eukaryotic cell that which has been modified to overexpress amyloid beta protein, APP, BACE1 and/or presenilin.

The object underlying the invention is completely achieved in this way.

Although liver is the main peripheral organ responsible for generation and degradation of Amyloid beta (Aβ) peptides, little is known about the role of Aβ in healthy and cirrhotic liver. In the present invention, it has been found that the generation and degradation of Aβ are compromised during human and rodent cirrhosis, which is reflected by the dramatic decrease of Aβ fragments and enzymes involved in its generation (BACE1 and gamma secretase presenilin).

In Alzheimer's disease, Aβ burden leads to increased permeability of brain capillaries. The results of the present invention shows that the large amount of Aβ produced by the liver upon physiologic condition serve the same function in regulating the permeability of liver sinusoids. The exposure of liver sinusoidal endothelial cells to Aβ leads to the increase in expression of liver permeability markers VEGF (vascular endothelial growth factor) and eNOS (endothelial nitric oxide synthase).

Further, with the present invention, the previously unknown high therapeutic potential of Aβ, as well as enzymes that are crucial for its generation and degradation in the liver cirrhosis is shown. According to the present invention, Aβ, due to its multifaceted anti-fibrogenic effects, efficiently counteracts the two major hallmarks of liver fibrosis/cirrhosis, i.e. 1) the activation of hepatic stellate cells and 2) the decreased liver endothelial permeability.

This finding has been proven by demonstrating the capacity of Aβ to downregulate the markers of HSC activation (alpha SMA, collagen type 1) and to upregulate the markers of liver sinusoidal endothelial cells permeability VEGF and eNOS. Aβ inhibits the key player of fibrosis/cirrhosis TGF-beta in activated HSC and in human liver endothelial cells (hLSEC).

Furthermore, the data of the present invention show that by treatment with Aβ, the hepatic expression of endothelial nitric oxide synthase (eNOS) could be increased. This could be shown in transgenic mouse models of Alzheimers diseases, having Amyloid precursor protein overexpressed and presenilin mutated in the brain, leading to an increase of systemic Aβ. With a high systemic and/or intrahepatic level of Aβ, the development and/or progression of liver diseases, e.g. liver cirrhosis, can be prevented or treated.

Within the present invention, it has also been shown that an inhibitor of neprilysin (NEP) can be employed as an amyloid beta related protein; neprilysin is an Aβ-degrading enzyme, and in activated HSC, the level of neprilysin is increased leading to a rapid uptake and degradation of Aβ. Therefore, according to the invention, a neprilysin inhibitor, such as, e.g. sacubitril, can be rapidly employed to treat and/or prevent liver diseases, in particular liver cirrhosis.

Further, and again as stated above, within the context of the present invention, the capacity of astrocytes to degrade Aβ to the lesser extent than HSC has been shown; due to the fact that eukaryotic cells like astrocytes enhance their capacity to produce Aβ in response to TGF-β and TNF-α, which are upregulated during cirrhosis, the finding of the inventors leads to the concept of treating liver diseases such as cirrhosis/fibrosis with eukaryotic cells that respond to the hallmarks of liver cirrhosis (TGF-β and TNF-a) with production of Aβ, exemplified by astrocytes.

In this context, and within the present invention, “a liver disease”—also called hepatic disease—means a type of damage to or disease of the liver. Preferably, the liver disease is liver cirrhosis or fibrosis. Also, preferably, the liver disease is hepatitis, including viral hepatitis, alcoholic hepatitis, autoimmune hepatitis, alcohol liver disease, fatty liver disease, nonalcoholic steatohepatitis, hepatocellular carcinoma or primary biliary cirrhosis.

In this context, “an amyloid beta related protein” is a protein related to the amyloid beta protein, and can, thus, be a protein represented by the amyloid beta protein, or derived from the amyloid beta protein, e.g. a fragment thereof, in particular fragments comprising 36, 37, 38, 39, 40, 41, 42, or 43 amino acids of the amyloid beta protein, or can be a protein involved with the generation/degradation of the amyloid beta protein. The amyloid beta protein is also known as Aβ or Abeta. Amyloid beta protein/peptides is/are derived from the amyloid precursor protein (APP), which is cleaved by beta secretase and gamma secretase to yield Aβ. Aβ molecules can aggregate to form flexible soluble oligomers which may exist in several forms.

By “Amyloid precursor protein” or “APP” is herein understood as an integral membrane protein expressed in many tissues and concentrated in the synapses of neurons. APP is known as the precursor molecule whose proteolysis generates beta amyloid (Aβ).

In a preferred embodiment, the amyloid beta peptide derived from the amyloid beta protein is selected from the group consisting of amyloid beta 40, amyloid beta 42 and amyloid beta 38.

AR is formed after sequential cleavage of the amyloid precursor protein (APP), a transmembrane glycoprotein of undetermined function. APP can be cleaved by the proteolytic enzymes α-, β- and γ-secretase; Aβ protein is generated by successive action of the β and γ secretases. The γ secretase, which produces the C-terminal end of the Aβ peptide, cleaves within the transmembrane region of APP and can generate a number of isoforms of 30-51 amino acid residues in length. Some of these isoforms are amyloid beta 40 (Aβ40), amyloid beta 42 (Aβ42) and amyloid beta 38 (Aβ38);

Further, in another embodiment of the compound according to the invention the compound involved in the generation of an amyloid beta peptide from APP is an enzyme selected from alpha-, beta- (BACE1), gamma-secretases, preferably presenilin.

Alpha secretases are a family of proteolytic enzymes that cleave amyloid precursor protein (APP) in its transmembrane region. The alpha-secretase pathway is the predominant APP processing pathway. Thus, alpha-secretase cleavage precludes amyloid beta formation and is considered to be part of the non-amyloidogenic pathway in APP processing. Upon cleavage by alpha secretases, APP releases its extracellular domain—a fragment known as APPsα—into the extracellular environment in a process known as ectodomain shedding.

Beta-secretase 1 (BACE1), also known as beta-site amyloid precursor protein cleaving enzyme 1, beta-site APP cleaving enzyme 1, membrane-associated aspartic protease 2, memapsin-2, aspartyl protease 2, and ASP2, is an enzyme that in humans is encoded by the BACE1 gene. Extracellular cleavage of APP by BACE1 creates a soluble extracellular fragment and a cell membrane-bound fragment referred to as C99. Cleavage of C99 within its transmembrane domain by γ-secretase releases the intracellular domain of APP and produces amyloid-β. Since gamma-secretase cleaves APP closer to the cell membrane than BACE1 does, it removes a fragment of the amyloid-βpeptide. Initial cleavage of APP by α-secretase rather than BACE1 prevents eventual generation of amyloid-β.

Gamma secretase is a multi-subunit protease complex, itself an integral membrane protein, that cleaves single-pass transmembrane proteins at residues within the transmembrane domain. Proteases of this type are known as intramembrane proteases. The most well-known substrate of gamma secretase is amyloid precursor protein, a large integral membrane protein that, when cleaved by both gamma and beta secretase, produces a short 42 amino acid peptide called amyloid beta. Presenilins are a family of related multi-pass transmembrane proteins which constitute the catalytic subunits of the gamma-secretase intramembrane protease complex.

Further, in another embodiment of the compound according to the invention the compound inhibiting the degradation of the amyloid beta protein, or of amyloid peptides derived therefrom, is an inhibitor of the enzyme neprilysin.

As discussed above, Neprilysin, also known as membrane metallo-endopeptidase (MME), neutral endopeptidase (NEP), cluster of differentiation 10 (CD10), and common acute lymphoblastic leukemia antigen (CALLA), is an enzyme that in humans is encoded by the MME gene. Neprilysin is a zinc-dependent metalloprotease that cleaves peptides at the amino side of hydrophobic residues and inactivates several peptide hormones including glucagon, enkephalins, substance P, neurotensin, oxytocin, and bradykinin. It also degrades the amyloid beta peptide. Inhibitors of neprilysin have been designed with the aim of developing analgesic and antihypertensive agents that act by preventing neprilysin's activity against signaling peptides such as enkephalins, substance P, endothelin, and atrial natriuretic peptide. Known inhibitors of the enzyme of neprilysin, which—accordingly—can be employed within the present invention, are Sacubitril/valsartan (Entresto/LCZ696), Sacubitril (AHU-377), a prodrug which is a component of sacubitril/valsartan, Sacubitrilat (LBQ657), the active form of sacubitril, RB-101, an enkephalinase inhibitor and UK-414,495, etc.

As discussed above, in a preferred embodiment of the genetically modified eukaryotic cell or the eukaryotic cells without genetic modification, which are naturally programmed to degrade Aβ with a less capability than hepatic stellate cells (HSC) in the liver, which is employed—according to the invention—for use in the treatment of liver diseases, is selected from a mesenchymal stromal cell, astrocytes, iPS-derived astrocytes, and somatic cells directly reprogrammed to astrocytes.

Mesenchymal stromal cells (MSCs) are spindle shaped plastic-adherent cells isolated from bone marrow, adipose, and other tissue sources, with multipotent differentiation capacity in vitro.

Astrocytes derived from iPS-(induced pluripotent stem cells) are, as such, as well as their generation, known in the art (see, e.g., Perriot S. et al., “Induced Pluripotent Stem Cell-Derived Astrocytes Are Differentially Activated by Multiple Sclerosis-Associated Cytokines”, Stem Cell Reports, 2018, 13;11(5):1199-1210) and can be retrieved accordingly. Same applies for the generation of astrocytes by reprogramming somatic cells (see, e.g., Lujan E. et al., “Direct conversion of mouse fibroblasts to self-renewing, tripotent neural precursor cells”, Proc. Natl. Acad. Sci. USA, 2012, 14; 109(7):2527-32).

The present invention accordingly also relates to a pharmaceutical composition for use in the treatment of a liver disease, especially liver fibrosis or cirrhosis, the pharmaceutical composition comprising an amyloid beta related protein, the amyloid beta related protein being selected from the group consisting of amyloid beta protein or amyloid beta peptides derived therefrom, amyloid precursor protein (APP), an enzyme involved in the generation of an amyloid beta peptide from APP, or an inhibitor of the degradation of amyloid beta protein or of amyloid beta peptides derived therefrom, and/or comprising a genetically modified eukaryotic cell that which has been modified to overexpress amyloid beta protein, APP, BACE1 and/or presenilin, together with an pharmaceutically acceptable excipient. The pharmaceutical composition may comprise a pharmaceutically acceptable carrier.

Presently, and as generally understood in the field, a “pharmaceutically acceptable carrier” is understood to mean any excipient, additive, or vehicle that is typically used in the field of the treatment of the mentioned diseases and which simplifies or enables the administration of the product according to the invention to a living being, and/or improves its stability and/or activity. The pharmaceutical composition can also incorporate binding agents, diluting agents or lubricants. The selection of a pharmaceutical carrier or other additives can be made on the basis of the intended administration route and standard pharmaceutical practice. As pharmaceutical acceptable carrier use can be made of solvents, extenders, or other liquid binding media such as dispersing or suspending agents, surfactant, isotonic agents, spreaders or emulsifiers, preservatives, encapsulating agents, solid binding media, depending upon what is best suited for the respective dose regime and is likewise compatible with the compound according to the invention. An overview of such additional ingredients can be found in, for example, Rowe (Ed.) et al.: Handbook of Pharmaceutical Excipients, 7th edition, 2012, Pharmaceutical Press.

As mentioned above, the present invention also relates to a method for treating or preventing a liver disease, the method comprising the step of administering to a subject in need thereof a pharmaceutically effective amount of a compound as detailed above and as claimed, or the genetically modified eukaryotic cell as detailed above or as claimed, or the pharmaceutical composition as detailed above, thereby treating or preventing the liver disease.

Further advantages are evident from the attached description and the figures and tables.

It will be appreciated that the features mentioned above and the features yet to be explained below are usable not only in the particular specified combination, but also in other combinations or alone, without departing from the scope of the present invention.

### BRIEF DESCRIPTION OF THE DRAWINGS

An embodiment of the invention is illustrated in the drawing and will be described in more detail below with respect to this.

In the Figures,

FIG. 1A-E show the Aβ degrading capacities of activated HSC. FIG. 1A shows double staining for NEP and α-SMA of M1-4HSC and HSC-T6 (day 2 after passage). Cell nuclei are stained with DAPI, 4′,6-diamidino-2-phenylindole. FIG. 1B shows an ELISA of Aβ42 shows higher capacity of M1-4HSC and HSC-T6 (n=4 in each group) for Aβ42 uptake vs. control samples (without cells) and vs. APC. FIG. 1C shows the uptake of Aβ42 by LX-2 increased with the number of cells (50,000-200,000). FIG. 1D and FIG. 1E shows degradation of Aβ42 and Aβ40 by M1-4HSC lysates (n=5) assessed by ELISA;

FIG. 2A and 2B show Western blots of the comparison of the content of NEP in lysates of HSC-T6 (FIG. 2A) and M1-4HSC (FIG. 2B) to astroglial primary culture (APC). Also the immune reaction of α-SMA in lysates of M1-4HSC;

FIG. 3A-3C show the influence of Aβ42 and Aβ40 on the level of α-SMA and TGF-β in M1-4HSC shown by Western Blots. FIG. 3A shows the treatment of M1-4HSC with 1000 pg/ml Aβ42. FIG. 3B shows the treatment of M1-4HSC with 1000 pg/ml Aβ40. FIG. 3C shows TGF-β from untreated M1-4HSC (ctrl.), cells treated with Aβ40 and Aβ42;

FIG. 4A-4D show the expression of NEP in the livers of BDL rats and mice and its correlation with α-SMA and GFAP. FIG. 4A shows RT-PCR analysis showing increased NEP and α-SMA mRNA and decreased GFAP mRNA in the livers of BDL rats (n=5, black bars) vs. respective sham operated (SO) controls (white bars, n=5). FIG. 4B and 4C show Western blots and densitometric calculations showing up-regulation of NEP in rat (n=3, FIG. 4B) and mouse (n=3, FIG. 4C) BDL livers vs. respective SO controls. FIG. 4D shows co-staining for NEP and GFAP (upper row), for NEP and α-SMA (middle row), for NEP and desmin in (lower row) in SO and BDL rat liver;

FIG. 5A-5D show the double staining of BDL and SO rat liver sections for marker proteins of HSC differentiation. FIG. 5A and 5B shows double staining of liver sections for GFAP and u-SMA. FIG. 5C and 5D shows double staining of liver sections for GFAP and desmin;

FIG. 6A-6C show the Western blot analyses of proteins involved in generation and degradation of Aβ peptides in SO and BDL rat liver. FIG. 6A shows pattern and densitometric calculations of APP degradation in BDL (black bars) and SO (white bars) rat livers (n=6). FIG. 6B shows the BACE endoproteolysis in BDL and SO rats (n=6). Decreased intensity of 55 and 25 kDa fragments whereas increased 35 kDa BACE fragment in BDL vs. SO rats. FIG. 6C shows the PS1 endoproteolysis in BDL and SO rats (n=6);

FIG. 7A-7F show the differences in the levels of Aβ-associated proteins in diseased and normal human liver samples. FIG. 7A shows qPCR analyses of fibrotic (hFL, grey bars, n=10) and cirrhotic (hCL, black bars, n=10) vs normal (hNL, white bars, n=9) human liver tissues. FIG. 7B-F show densitometric calculations and images of immunoreactive bands in Western blots of hCL vs. hNL (n=6) showing: FIG. 7B down-regulation of 100, 30 and 10 kDa APP fragments in hCL FIG. 7C upregulation of mature 70 kDa and down-regulation of 37, 27 and 20 kDa BACE fragments in hCL vs. hNL, FIG. 7D up-regulation of mature 70 kDa and downregulation of immature 55 and 27 kDa PS1 fragments in hCL vs. hNL; FIG. 7E Down-regulation of NEP in hCL; FIG. 7F down-regulation of myelin basic protein (MBP) in hCL vs. hNL;

FIGS. 8A-[E]8F show the Aβ fragments and eNOS in cirrhotic liver and in-vitro effect of Aβ on Col-1 and eNOS. FIG. 8A shows down-regulation of A1340, Aβ42 and Aβ38 in hCL (black bars, n=9) vs. hNL (white bars, n=9). FIG. 8B shows down- regulation of Aβ42 in rat BDL (n=4) and mouse BDL (n=4) versus respective SO (n=4). FIG. 8C shows decreased eNOS in hCL vs. hNL (n=5). FIG. 8D shows reduction of eNOS in rBDL vs. rNL (n=4). FIG. 8E shows upregulation of eNOS synthesis by hSEC under treatment with Aβ42 (n=6). FIG. 8F shows suppression of Col-1 production in M1-4HSC (n=6); and

FIGS. 9A-9B show the inhibition of Aβ40 and 42 degradation by HSC in response to sacubitrilat (LBQ657). FIG. 9A shows inhibition of Aβ40 degradation. FIG. 9b shows inhibition of Aβ42 degradation.

### EMBODIMENTS

### EXAMPLES

### Materials and Methods

### Human Liver Tissue Samples

Human liver tissues were obtained from 44 patients comprising 21 males and 23 females (15 patients with normal liver, 15 with fibrosis and 14 with cirrhosis).

### Animal Experiments

For the bile duct ligation (BDL), Sprague Dawley rats and C56BL/6J mice (Charles River, Sulzfeld, Germany) were used. As a model of Alzheimer's disease double transgenic mice B603-Tg(APPswe, PSEN 1dE9)85Dbo/J (APP/PS1 mice) were purchased from Jackson Laboratories (Bar Harbor, Me., USA).

### Cell Culture

M1-4HSC cell line was provided. Rat HSC-T6 and the human HSC line have been previously described (Vogel S, Piantedosi R, Frank J et al. An immortalized rat liver stellate cell line (HSC-T6): a new cell model for the study of retinoid metabolism in vitro. J Lipid Res 2000; 41:882-893 and Xu L, Hui A Y, Albanis E et al. Human hepatic stellate cell lines, LX-1 and LX-2: new tools for analysis of hepatic fibrosis. Gut 2005; 54:142-151.).

Astroglia-rich primary cultures (APC) were prepared from newborn C57/BL6 (Charles River) mouse brains as described elsewhere (Lourhmati A, Buniatian G H, Paul C, et al. Age-dependent astroglial vulnerability to hypoxia and glutamate: the role for erythropoietin. PLoS One 2013; 8:e77182.). Briefly, the cells obtained from 5-7 brains of newborn littermates were mechanically dissociated, centrifuged and plated onto cell culture flasks (1×106 cells/75 cm2) in DMEM with 4.5 g/l Glucose supplemented with 10% foetal calf serum, 100 μg/ml streptomycin sulphate, 100 units/ml penicillin G and 1 μM pyruvate (Biochrom AG, Berlin, Germany) in a humidified 10% CO2 atmosphere at 37° C.

HSC-T6 were grown in DMEM with 4.5 g/l Glucose supplemented with 10% foetal calf serum, 100 μg/ml streptomycin sulphate, 100 units/ml penicillin G and 1 μM pyruvate in a humidified 10% CO2 atmosphere at 37° C.

M1-4HSC, human hepatic sinusoidal endothelial cells-SV40 (HSEC, Applied Biological Materials, Richmond, BC, Canada) and LX-2 cells were grown in DMEM high with 4.5 g/l Glucose containing either 2% (for LX-2), 5% (for HSEC) or 10% foetal calf serum (for M14HSC), 1% non-essential amino acids (only for M1-HSC), 100 U/ml penicillin and 100 μg/ml streptomycin (only for HSEC, Gibco, Thermo Fisher, Darmstadt, Germany). Cells were kept at 37° C. in an atmosphere containing either 5% (for M1-4HSC and HSEC) or 10% CO2 (for LX-2).

### Aβ Quantification in Cell Cultures

For the comparison of different cell types regarding their ability to utilise Aβ42, M1-4HSC, HSC-T6, LX-2 and astroglial primary cultures (APC) were incubated with medium containing synthetic Aβ. Adherent cells (50,000 or 100,000 cells/well in 24-well plates) were incubated with medium containing 1000 pg/ml of synthetic Aβ42. After 24 h, supernatant was centrifuged at 1500 g for 15 minutes and frozen at −80° C. until analysis.

The data revealed a significant loss (over 50%) of Aβ 24-48 h after incubation with cell culture medium in the absence of cells (not shown), which can be ascribed to natural degradation, adhesion to the polystyrene plates and/or spontaneous formation of Aβ oligomers or polymers (Ahmed M, Davis J, Aucoin D, et al. Structural conversion of neurotoxic amyloid-beta(1-42) oligomers to fibrils. Nat Struct Mol Biol. 2010; 17:561-567.). Therefore, as a control for inherent decrease of amyloid concentrations control samples containing only culture medium, without cells (w/c) were incubated with Aβ for the same periods of time.

To quantify the Aβ42-degading ability of M1-4HSC lysate's, cell lysates from M1-4HSC (50,000 cells/ml) were obtained by 2 freezing thawing cycles at −80° C. and centrifugation for 10 min at 20,000 g. Lysates were incubated with DMEM containing 1000 pg/ml of synthetic Aβ42 and Aβ40 in presence or absence of 5 mM EGTA for 30 or 60 minutes.

Aβ42 and Aβ40 were measured with the human Aβ EZHS ELISA Kit (Merck Millipore, Darmstadt, Germany) according to the manufacturer's protocol.

### Quantification of Aβ Peptides in Liver and Brain Homogenates

Human liver tissue was homogenized in ice cold Lysis Buffer (300 mM NaCl, 50 mM Tris, 2 mM MgCl2, containing ‘Mini Complete Protease Inhibitors’, (Sigma-Aldrich, Taufkirchen, Germany)). Rat liver tissue from BDL and SO rats was homogenized in 4 volumes of cold 6.25M guanidine HCl in 50 mM Tris buffer pH 8.0. Protein concentrations were determined using the Detergent Compatible (DC). Protein assay (Bio-Rad, Hercules, Calif.). Tissue lysates were centrifuged at 20,000 g for 10 minutes at 4° C.

Liver Aβ38/40/42 fragments were detected by V-Plex® Kit (Mesoscale, Rockville, Md.) using the Aβ antibody (4G8) recognizing human and rodent Aβ40, Aβ42 and Aβ38. In healthy and cirrhotic human liver samples Aβ40 was quantified also by EZBrain ELISA Kit (Merck Millipore, Darmstadt, Germany) according to the manufacturer's protocol.

Brain homogenates from APP/PS1 and WT mice were analyzed by human Amyloid β42 EZBrain ELISA Kit according to the manufacturers protocol.

### Statistical Analyses

All data presented in this study were analyzed by One-way ANOVA analysis with post hoc Bonferroni's multiple comparison test or Student's t-tests for single comparisons and by employing GraphPad Prism Software (GraphPad Software Inc, La Jolla, Calif.). p<0.05 was considered significant.

### Results

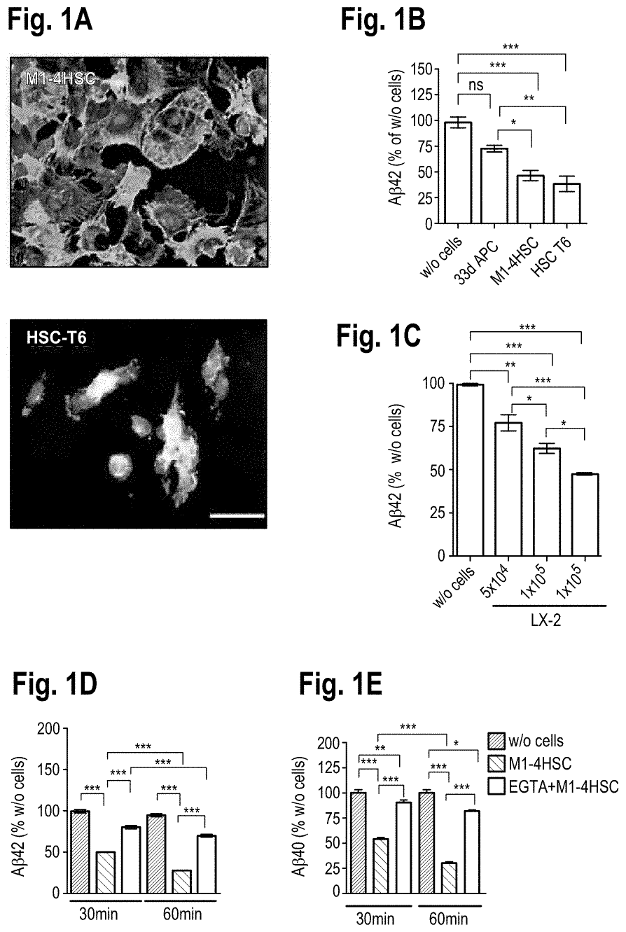

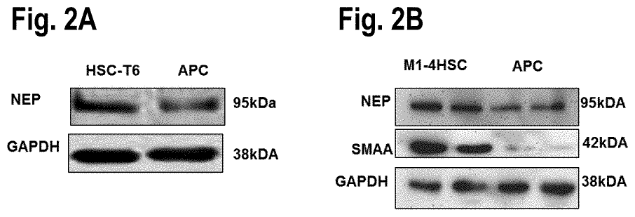

The AR-degrading enzyme NEP was demonstrated in M1-4HSC and HSC-T6 cells by immunofluorescence (FIG. 1A) and Western blot (FIG. 2A and B). Based on Western blot, M1-4HSC (FIG. 2B) and HSC T6 cells (FIG. 2A) contained more NEP than APC. To further explore the similarities between HSC and astrocytes we next compared the capacity of HSC cell lines to eliminate Aβ from the 10 environment to astrocytes.

The results of the experiment are shown in FIG. 1A to FIG. 1E. FIG. 1A shows double staining for NEP and α-SMA of M1-4HSC and HSC-T6 (day 2 after passage). Cell nuclei are stained with DAPI, 4′,6-diamidino-2-phenylindole. FIG. 1B ELISA of Aβ42 shows higher capacity of M1-4HSC and HSC-T6 (n=4 in each group) for Aβ42 uptake vs. control samples (without cells) and vs. APC. FIG. 1C shows the uptake of Aβ42 by LX-2 increased with the number of cells (50,000-200,000). FIG. 1D and FIG. 1E shows degradation of Aβ42 and Aβ40 by M1-4HSC lysates (n=5) assessed by ELISA. Nearly identical time-dependent degradation of Aβ42 and Aβ40 (grey bars in FIG. 1D and FIG. 1E) by 50% after 30 minutes and by 75% after 60 minutes. The degradation of both Aβ fragments by M1-4HSC lysates was dramatically inhibited in the presence of EGTA (cf. grey and white bars in FIG. 1D and FIG. 1E). The concentrations of Aβ42/40 in cell-containing samples were normalized to their respective time-specific standard (control without cells, black bars). The data are shown as means±SEM, *p<0.05, **p <0.01, ***p<0.001, respectively.

Immunofluorescence demonstrated the presence of NEP in both M1-4HSC and HSC-T6 cells (FIG. 1A). A two-fold higher uptake of Aβ42 (reflected by its decrease in cell culture supernatant) by M1-4HCS and HSC-T6 vs. APC was determined by ELISA (FIG. 1B). From 1000 pg/ml of Aβ42 supplemented to the culture medium, about 50% of the peptide was absorbed on plastic carrier after 48 h of incubation. From the remaining part of Aβ available for cells in culture medium 28%, 55% and 60% of the peptide were internalized by astrocytes, M1-4HSC and HSC-T6 cells, respectively (FIG. 1B). The amount of Aβ42 internalized by LX-2 grew with increasing number of cells in culture (FIG. 1C).

To investigate whether Aβ simply accumulated or underwent degradation by the cells, Aβ42 (FIG. 1A) or Aβ40 was added to lysates of M1-4HSC. After 30 and 60 minutes of incubation, the level of Aβ42 and Aβ40 was reduced to 50% and 25% respectively, compared to the initial level in control samples without cell lysates (cf. grey bars with black bars in FIG. 1D and FIG. 1E). To confirm that the disappearance of Aβ from HSC lysates reflects its enzymatic degradation rather than non-specific loss that can occur in cell lysates the activity of zinc-dependent AR-degrading enzymes comprising angiotensin converting enzyme (ACE) and endothelin converting enzyme (ECE) known to be present in HSC1 as well as NEP was blocked by 5 mM EGTA (white bars in FIG. 1D and FIG. 1E). Incubation of M1-4HSC lysates for 30 and 60 minutes with EGTA abolished the degradation of Aβ40/42 by the cell lysates (cf. white bars with grey bars in FIG. 1A, FIG. 1B).

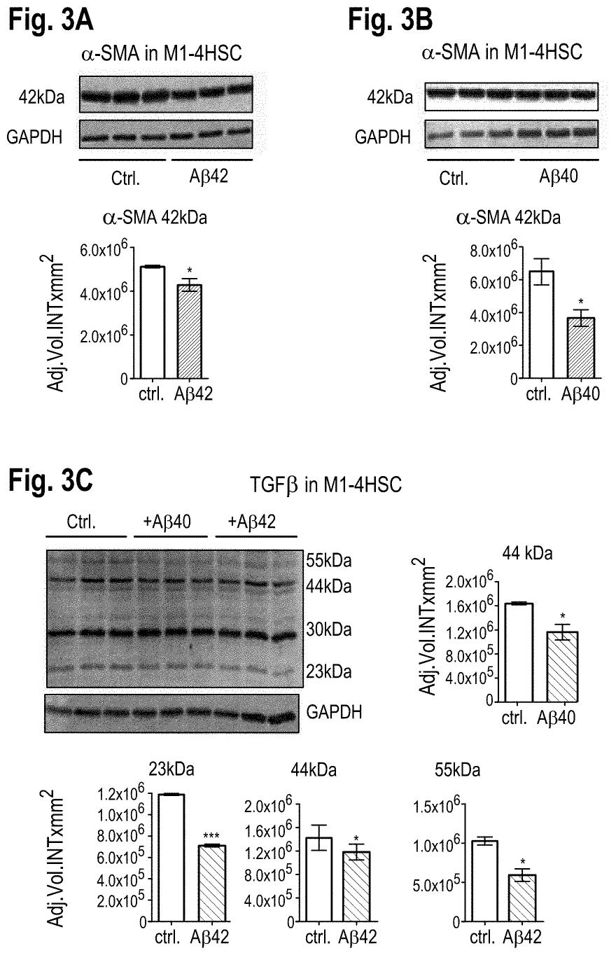

The results shown in FIG. 3A to C it can be seen that the treatment of M1-4HSC with both Aβ42 and Aβ40 reduced the expression of α-SMA (FIG. 3A and B) and TGF-β (FIG. 3C). Suppression of TGF-β synthesis by Aβ42 was more potent than Aβ40 (FIG. 3C).

FIG. 3A and 3B shows the treatment of M1-4HSC with 1000 pg/ml Aβ42 (FIG. 3A) and Aβ40 (FIG. 3B). FIG. 3C shows TGF-β from untreated M1-4HSC (ctrl.), cells treated with Aβ40 and Aβ42. Densitometric calculations showing significantly decreased intensity adjusted to the respective GAPDH lane (Adj.V0I. INT×mm2) of 55 kDa, 44 kDa and 23 kDa bands in cells treated with Aβ42. In contrast, Aβ40 was capable of decreasing only the 44 kDa band. The data are shown as means±SEM, *p<0.05, ***p<0.001.

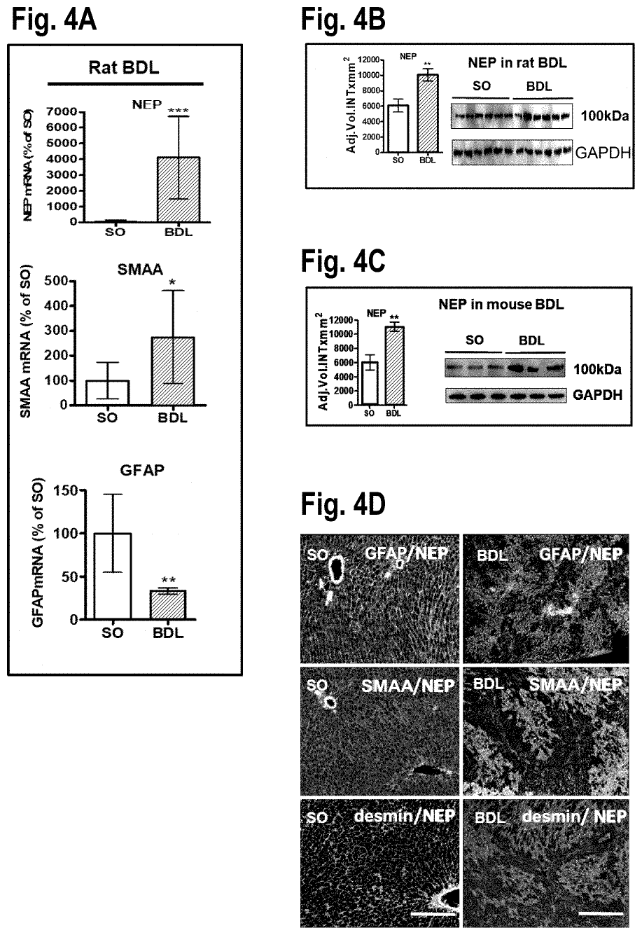

FIG. 4 shows the expression of NEP in the livers of BDL rats and mice and its correlation with α-SMA and GFAP. FIG. 4A shows RT-PCR analysis showing increased NEP and α-SMA mRNA and decreased GFAP mRNA in the livers of BDL rats (n=5, black bars) vs. respective sham operated (SO) controls (white bars, n=5). FIG. 4B and C show Western blots and densitometric calculations showing up-regulation of NEP in rat (n=3, FIG. 4B) and mouse (n=3, FIG. 4C) BDL livers vs. respective SO controls. FIG. 4D shows co-staining for NEP and GFAP (upper row), for NEP and α-SMA (middle row), for NEP and desmin in (lower row) in SO and BDL rat liver. Cell nuclei are stained with DAPI. Scale bar: for the left row 200 μm, for the right row 500 μm.

RT-PCR analyses of whole liver RNA demonstrated significant up-regulation of NEP and α-SMA mRNA concomitant with a decrease of GFAP mRNA in BDL vs. SO rat livers (FIG. 4A). These results were confirmed by Western blot of NEP in rat (FIG. 4B) and mouse BDL (FIG. 4C) and SO livers. Also, stronger reaction of NEP in liver sections of rat BDL vs. respective SO was apparent by immunofluorescence of NEP and α-SMA (upper row in FIG. 4C) or NEP and GFAP (middle row in FIG. 4C) or NEP and desmin (lower row in FIG. 4C).

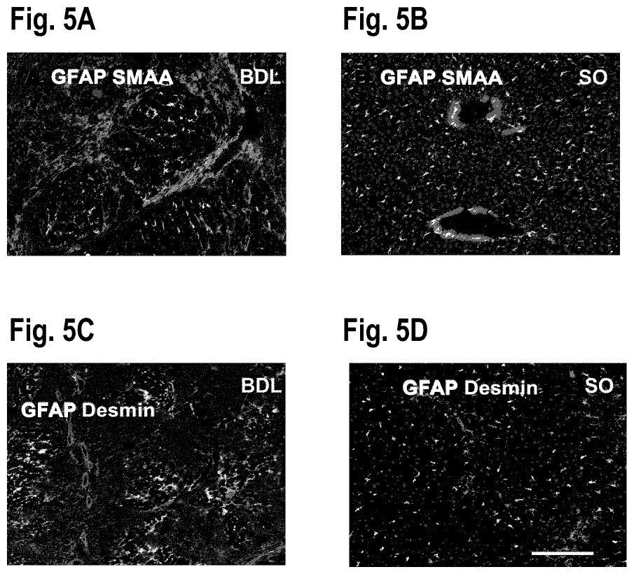

Because HSC are the main cell type involved in BDL-induced cirrhosis, the inventors first investigated changes in their phenotype in BDL vs. SO under staining for established marker proteins of HSC: GFAP, α-SMA and desmin. The results can be seen in FIG. 5A-D.

FIG. 5A-D shows the double staining of BDL and SO rat liver sections for marker proteins of HSC differentiation. FIG. 5A and 5B shows double staining of liver sections for GFAP and u-SMA. FIG. 5C and 5D shows double staining of liver sections for GFAP and desmin. GFAP was visualized using FITC-conjugated goat anti-rabbit IgG whereas α-SMA and desmin using Cy3-conjugated goat anti-mouse IgG. Scale bar in FIG. 5A-D is 200 μm.

In FIG. 5A vs. 5B and 5C vs. 5D it can be seen that in BDL there were large areas containing cells solely expressing α-SMA or desmin, as well as regions containing cells expressing GFAP and/or desmin (FIG. 5C and 5D).

Double labelling of BDL and SO rat liver sections with (α-SMA and NEP antibodies (upper row of FIG. 4D) localized NEP+ cells to cirrhotic nodules. Most of these cells expressed both α-SMA and NEP, which appeared as intense yellow staining. A large population of HSC solely expressed α-SMA or NEP in cirrhotic areas and in the regions surrounding the nodules. In contrast, in SO rat liver sections, NEP and α-SMA were co-localized only within the vascular wall. In BDL rat liver the cirrhotic nodules enriched by GFAP-negative A-HSC strongly expressed NEP (see FIG. 4D). Most cells residing between regenerative nodules were NEP-negative and solely expressed GFAP (see FIG. 4D). However, BDL rat liver contained also regions enriched with cells co-expressing GFAP and NEP, most likely reflecting the transitional state of HSC activation (see FIG. 5A). This contrasted to SO rat liver in which NEP was solely expressed in the membranes of hepatocytes throughout the liver parenchyma and was absent from quiescent HSC strongly expressing GFAP (see FIG. 4D FIG. 5B).

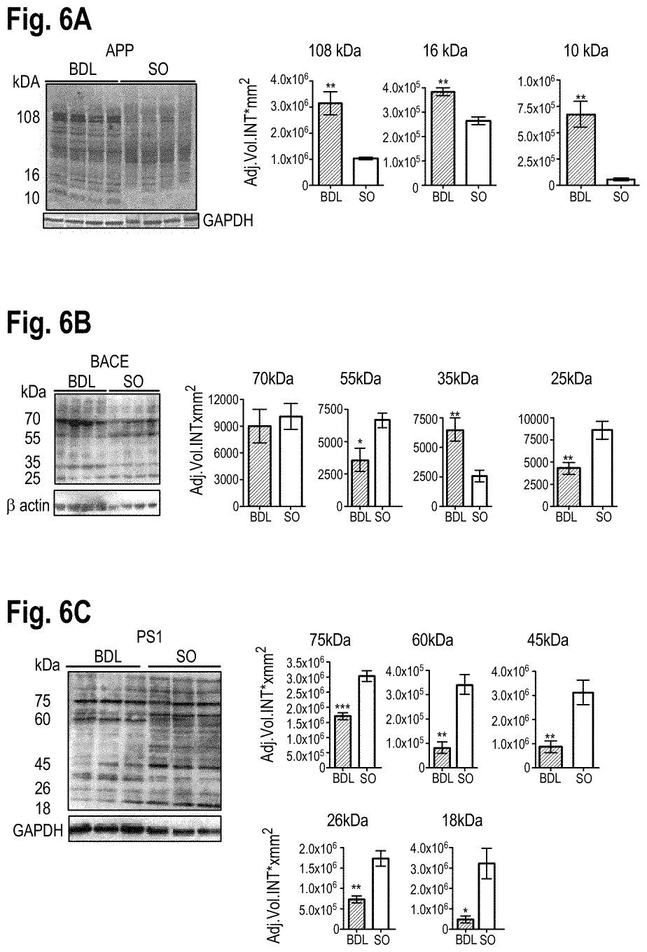

FIG. 6A-C show the Western blot analyses of proteins involved in generation and degradation of Aβ peptides in SO and BDL rat liver. Pattern and densitometric calculations of APP degradation in BDL (black bars) and SO (white bars) rat livers (n=6) can be seen in FIG. 6A. Increased intensity of 108, 16 and 10-11 kDa APP fragments in BDL vs. SO rats. FIG. 6B shows the BACE endoproteolysis in BDL and SO rats (n=6). Decreased intensity of 55 and 25 kDa fragments whereas increased 35 kDa BACE fragment in BDL vs. SO rats. FIG. 6C shows the PS1 endoproteolysis in BDL and SO rats (n=6). Decreased intensity of 75, 60 45, 26 and 18 kDa PS1 fragments in BDL vs. SO rats. Western blot images demonstrate 3-4 representative samples from each group out of n=6. The data are shown as means±SEM, *p<0.05, **p<0.01, ***p<0.001.

In BDL livers of rat (see FIG. 6) the amount of the non-amyloidogenic 108 kDa N-terminal APP fragment was higher compared to that detected in the livers of SO control animals (see FIG. 6A). Remarkably, also the 10 kDa APP fragment, which is a product of the BACE reaction, was significantly higher in BDL (see FIG. 6B). There was a general decrease of all BACE fragments in BDL rat liver, except for the 35 kDa fragment which was significantly increased in BDL rats (see FIG. 6B). This BACE fragment is strongly expressed in liver. Western blotting of PS1 (see FIG. 6C) in rat livers revealed five main PS1 fragments. All of them were significantly down-regulated in BDL vs. SO rat liver, including large mass 75 kDa, immature 60 kDa, 45 kDa, as well as mature 26 kDa N-terminal and 18 kDa C-terminal PS1 fragments.

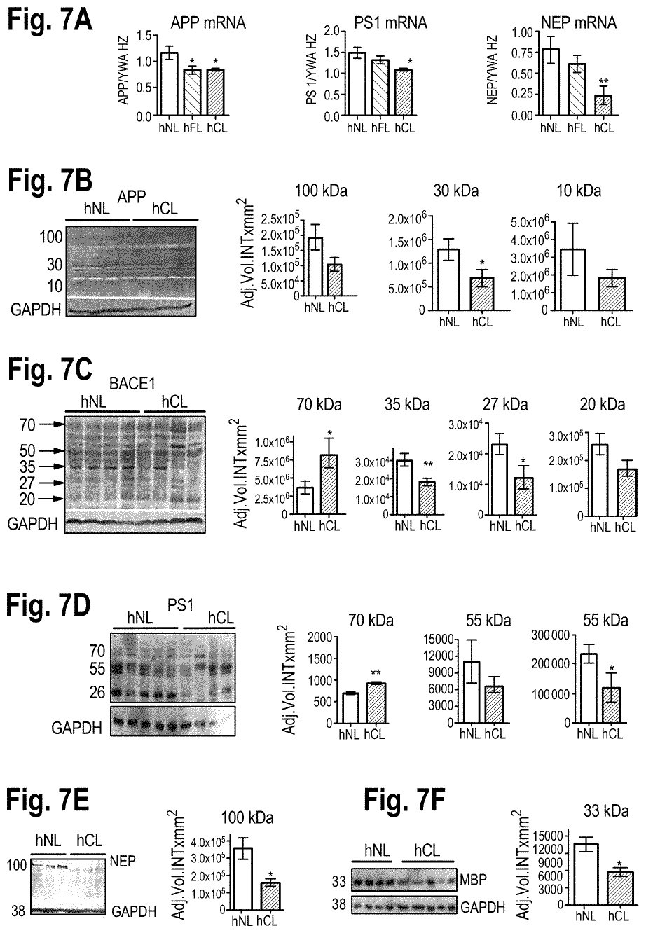

FIG. 7 shows differences in the levels of AR-associated proteins in diseased and normal human liver samples. FIG. 7A shows qPCR analyses of fibrotic (hFL, grey bars, n=10) and cirrhotic (hCL, black bars, n=10) vs normal (hNL, white bars, n=9) human liver tissues showing down-regulation of APP mRNA in hFL and hCL vs. hNL of PS1 NEP mRNA mRNA in hCL vs. hNL and of NEP mRNA in hFL and hCL vs. hNL. FIG. 7B-F show densitometric calculations and images of immunoreactive bands in Western blots of hCL vs. hNL (n=6) showing: FIG. 7B down-regulation of 100, 30 and 10 kDa APP fragments in hCL FIG. 7C up-regulation of mature 70 kDa and down-regulation of 37, 27 and 20 kDa BACE fragments in hCL vs. hNL, FIG. 7D up-regulation of mature 70 kDa and down-regulation of immature 55 and 27 kDa PS1 fragments in hCL vs. hNL; FIG. 7E Down-regulation of NEP in hCL; FIG. 7F down-regulation of myelin basic protein (MBP) in hCL vs. hNL. Western blot images are shown as 34 representative samples from a total of 6 probes analysed per group. Means±SEM, *p<0.05, **p<0.01, ***p<0.001.

The results of qPCR analyses of human liver species showed down-regulation of APP mRNA in hFL and hCL vs. hNL (FIG. 7A). Also PS1 mRNA was decreased in hCL vs. hNL. Strong down-regulation of NEP mRNA was observed in both hFL and hCL vs hNL (FIG. 7A). Western blotting showed uniform reduction of the 100 kDa, 30 kDa and 10 kDa APP fragments in hCL vs. hHL (FIG. 7B). Compared to hNL hCL the immune reaction of the mature 70 kDa BACE band was increased in hCL, whereas low mass bands of 35-37 kDa, 27 kDa and 20 kDa BACE fragments known as enzymatically active fragments were decreased in hCL compared hNL (FIG. 7D), These data indicate the amyloidogenic path of APP proteolysis in hNL. The densitometry of immune reactive PS1 bands showed up-regulation of the mature 70 kDa PS1 fragment on the expense of the immature 55 kDa PS1 followed by decline of the enzymatically active 26 kDa PS1 fragment (FIG. 7D). Western blot also showed down-regulation of NEP (FIG. 7E) and MBP (FIG. 7F) in hCL vs. hNL.

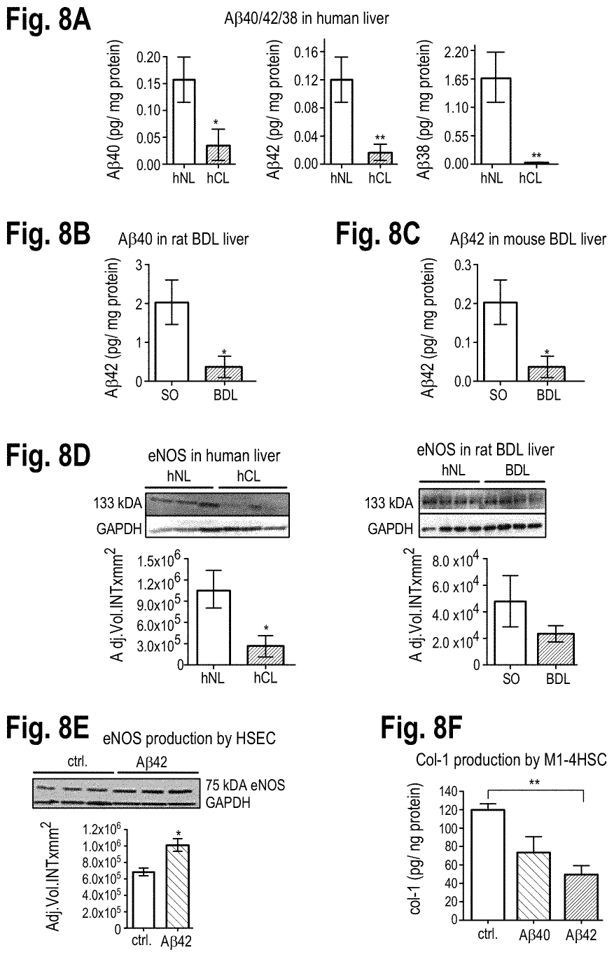

FIG. 8A-E shows Aβ fragments and eNOS in cirrhotic liver and in-vitro effect of Aβ on Col-1 and eNOS. In FIG. 8A it can be seen that V-PLEX® Aβ peptide panel with 4G8 Aβ antibody analyses show down-regulation of A1340, Aβ42 and Aβ38 in hCL (black bars, n=9) vs. hNL (white bars, n=9). In FIG. 8B it can be seen that down-regulation of Aβ42 in rat BDL (n=4) and mouse BDL (n=4) versus respective SO (n=4). Western blot analyses revealed: decreased eNOS in hCL vs. hNL (n=5) (see FIG. 8C); reduction of eNOS in rBDL vs. rNL (n=4) (see FIG. 8D); upregulation of eNOS synthesis by hSEC and suppression of Col-1 production in M1-4HSC (n=6) under treatment with Aβ42 shown by ELISA (see FIG. 8E and 8F). Means±SEM, *p<0.05, **p<0.01, ***p<0.001.

V-PLEX® analysis showed around 5-, 10- and 160-fold down regulation of Aβ40/42/38 peptides respectively in hCL vs. hNL (FIG. 8A), about 6 fold reduction of Aβ42 in BDL-induced rat (FIG. 8B) and BDL-induced mouse (FIG. 8C) cirrhosis vs. respective control livers. Down-regulation of Aβ40 in hCL vs. hNL was detected also by ELISA (not shown). Western blot analysis showed down-regulation of eNOS in human CL (FIG. 8D) and in rat BDL (FIG. 8E) vs. respective controls. In both, human CL and rat BDL no significant changes in the level of the neuron-specific isoform of NOS (nNOS) was detected (not shown). A correlation between Aβ and eNOS was also observed in the brains of a double transgenic model of Alzheimer's disease, APP/PS1 mice (not shown), characterized by altered BBB function. High levels of Aβ in these animals were concomitant to up-regulation of eNOS, demonstrating an opposite regulation of the permeability in liver and brain capillary. Treatment of human liver endothelial cells with Aβ resulted in increased synthesis of eNOS, nitric oxide producing enzyme (FIG. 8E) that is critical for the permeability of liver sinusoids. The permeability of liver sinusoids also depends on the level of collagen type 1 produced mainly by activated HSC resulting in collagenization of liver capillaries. Therefore, the inventors next investigated whether Aβ regulates the production of collagen1 by M1-4HSC; there was a greater than 2-fold decrease of the Col-1 level in M1-4HSC culture following treatment with Aβ42 and only a slight reduction influenced by Aβ40 (FIG. 8F).

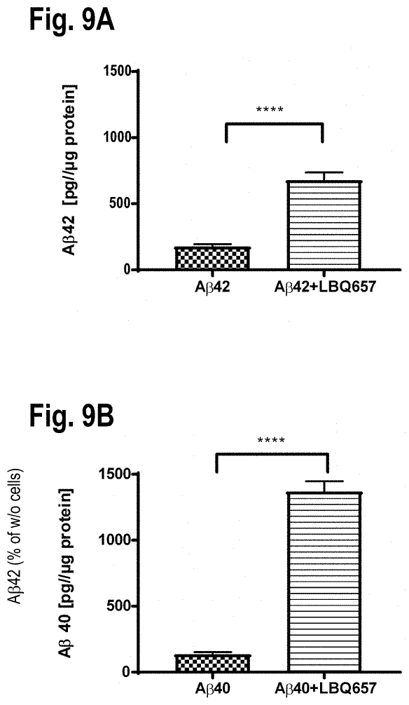

FIG. 9 (A-B) shown an inhibition of Aβ40 and 42 degradation by HSC in response to sacubitrilat (LBQ657). Primary murine HSC were incubated with 1000 pg/ml Aβ40 or 42 with and without LBQ657. The content of Aβ in the cell culture supernatant was measured by ELISA 48 h after incubation with Aβ40 or 42 with and without LBQ657. Decreased degradation of Aβ in HSC treated with LBQ657 is reflected by a higher content of Aβ in the cell culture supernatant.

### Discussion

Within the present invention, the down-regulation of Aβ-peptides in human and rodent cirrhosis is shown for the first time. In contrast to cirrhosis, in healthy human liver APP is processed via amyloidogenic proteolysis as demonstrated by Western blot showing:
i) low expression of large mass N-terminal APP-fragment produced via the alpha-secretase pathway;
ii) increased reaction of the enzymatically active 30-35 kDa BACE fragment previously detected in non-neural cells
iii) higher amounts of the 10-11 kDa C-terminal APP fragment generated by BACE, an enzyme initiating the first step of amylodogenic degradation of APP
iv) higher amounts of low mass PS1 derivatives known as enzymatically active and finally
v) significantly higher levels of Aβ42/40/38 peptides correlating with larger amounts of small carboxy-terminal 10-11 kDa APP fragment in hNL vs. hCL.

Further, within the present invention it was found that activated HSC can internalise and degrade Aβ-peptides, underscoring their role in the active elimination of Aβ from diseased liver. Furthermore, it was found out that A-HSC showed a higher potency for Aβ uptake, and they contained larger amounts of NEP in comparison with astrocytes. The degradation of Aβ40 and Aβ42 by M1-4HSC lysates was time-dependent and could be inhibited by EGTA, confirming the presence of an enzymatically active NEP, a zinc-dependent Aβ degrading enzyme. Treatment with EGTA might affect also the activity of other zinc-containing enzymes for example angiotensin converting enzyme (ACE) and endothelin converting enzyme (ECE) present in HSC. The results demonstrate that A-HSC establish a potent intrahepatic sink for amyloidogenic Aβ species during cirrhosis.

Aβ contributes to the maintenance of a quiescent phenotype of HSC known to regulate normal liver homeostasis. This is evidenced by suppressive effects of Aβ40 and Aβ42 on α-SMA synthesis in activated M1-4HSC, demonstrating a decreased α-SMA/GFAP ratio and reversal of HSC to a quiescent phenotype. In BDL-induced cirrhosis the reduction of Aβ is accompanied by down-regulation of GFAP mRNA. A similar effect of Aβ on up-regulation of GFAP has been observed after its intra-cerebro-ventricular injection into the mouse brain.

Activation and contraction of HSC in cirrhosis leads to increased extra-cellular matrix protein production leading to collagenization of the perisinusoidal space and transformation of the fenestrated hepatic sinusoids into continuous capillaries proper for cirrhosis. These ultrastructural changes limit blood-liver exchange and the hepatic flow. The anti-fibrogenic effects of Aβ as reflected in decreased production of TGF-β and Col-1 and reduced levels of α-SMA in HSC inhibit the development of cirrhosis and remodelling of blood-liver interface. These results evidence the importance of Aβ42 for liver-specific functions associated with the permeability of liver sinusoids. Interestingly in human cerebrovascular smooth muscle cells Aβ induced the degradation of α-SMA.

Liver perfusion is largely regulated by nitric oxide (NO), a powerful vasodilator produced by eNOS in hepatocytes and endothelial cells. The AR-induced effects on α-SMA, TGF-β and Col-1 synthesis by HSC shown here are true also for NO effects demonstrated in vivo: Thus, HSC targeted nanoparticle delivery of NO blocks collagen I, α-SMA and fibrogenic genes in rat livers affected by fibrosis and portal hypertension thereby it contributes to maintenance of the fenestrated construction of liver endothelial cells. In vitro, NO acts as a reactive oxygen species (ROS) scavenger, enhancing the accumulation of peroxynitrite and inhibiting the proliferation of HSC38. The effects of NO during neurological diseases characterized by Aβ accumulation are also partially mediated by peroxynitrite, which increases the permeability of the BBB.

The functional link between Aβ and NO can be inferred from the experiments by the inventors showing high levels of Aβ and eNOS in healthy liver and their reduction in cirrhosis. These results are demonstrated by the in vitro studies showing significantly elevated production of eNOS, thereby enhancing production of NO by Aβ42-treated hSEC. The results are consistent with in-vitro studies showing Aβ-stimulated production of NO in astrocytes.

While increased levels of NO and Aβ in the brain cause pathologic changes in brain-specific functions, high levels of Aβ in the liver cooperate with NO to support the physiologically essential permeability of liver sinusoids. In the light of studies demonstrating an Aβ-provoked decrease of tightjunction proteins in brain endothelial cells, the levels of Aβ in cirrhotic liver contribute to the loss of fenestrations and inhibit the generation of tight junctions and capillarization of hepatic sinusoids during cirrhosis. It is tempting to speculate that a high level of Aβ in healthy liver is important for the maintenance of fenestrated construction of liver capillaries. In addition, low levels of Aβ in cirrhotic liver may predispose the neuron-like differentiation of myofibroblast-like-HSC similar to young astrocytes in healthy brain.

Another key finding of the experiments by the inventors is that cirrhosis down-regulates MBP, the main component of myelin sheaths, which be considered as a marker of integrity of hepatic parenchymal nerves, which disappear during cirrhosis. Notably, purified human brain MBP and recombinant human MBP can degrade Aβ40 and Aβ42 in-vitro and reduce the area of parenchymal and cerebral vascular amyloid deposits in Tg2576 mouse brain sections. In-vitro studies showed that MBP mimics the effects of Aβ in that it strongly stimulates the production of NO via activation of iNOS in adult human astrocytes.

High levels of MBP, eNOS and Aβ in healthy liver vs. cirrhotic liver shown here also demonstrate their synergism in liver. Thus, it is pointed out that endothelial cell dysfunction during cirrhosis, characterized by poor permeability of liver sinusoids be at least partially caused by decreased levels of Aβ and MBP followed by down-regulation of eNOS. The experiments demonstrate decreased levels of eNOS in chronic human cirrhosis and in BDL model of cirrhosis.

Further, the experiments show significant down-regulation of NEP mRNA and protein in chronic human cirrhosis. Similar changes in NEP and MBP in chronic cirrhosis points to a minimal contribution of both proteins to the disappearance of Aβ in chronic human cirrhosis. The decrease of MBP and NEP upon cirrhosis underlie the intrinsic protective mechanisms for retaining at least minimal amounts of Aβ to upkeep the weakened liver functions.

In rat BDL models, processing of APP is characterized by production of larger amounts of non-amyloidogenic 108 kDa as well as amyloidogenic 16 kDa and 10 kDa APP fragments in BDL compared to SO. In rat BDL a higher amount of functionally mature 35 kDa fragment of BACE is accompanied by uniform decreases of all PS1 fragments resulting in down-regulation of Aβ.

In view of the capacity of NEP to degrade Aβ, the upregulation of NEP in the BDL model of cirrhosis amplify injury that is already promoted by low levels of PS1 and NO. High portal pressure in the BDL model of cirrhosis is caused mainly by increased levels of Angiotensin (Ang) II generated from Ang I and catalysed by ACE. The contribution of NEP to increased portal pressure was disproved by vasoconstrictory effects of thiorphan, the specific inhibitor of NEP. It has been shown that in BDL NEP contributes to generation of Ang-(1-7), a vasorelaxant which is increased in BDL and which counteracts the vasoconstrictory effects of ACE and Ang II.

The results demonstrate the following scenario and role for Aβ in liver-specific functions: In healthy liver hepatocytes produce large amounts of APP, BACE1 and PS1 resulting in generation and release of Aβ into the extracellular space in which Aβ shows different activities: it deactivates HSC that is illustrated by decreased levels of α-SMA, collagen and TGF-B. Thus the quiescent phenotype of HSC in healthy liver is at least partially supported by Aβ. In addition, Aβ induces the synthesis of NO (eNOS) by hSEC. Thus, Aβ may contribute to permeability of liver sinusoids via anti-fibrogenic effects on HSC and via induction of eNOS in hSEC. The activities of Aβ and eNOS in healthy liver are probably supported by a high level of MBP, a protein shown to mimic the effects of Aβ, i.e., increase the production of NO in astrocytes. Further Aβ-related “loss of function” experiments will be undertaken to evaluate the overall impact of Aβ on the permeability of liver sinusoids.

In contrast, in cirrhosis the decreased expression of APP, BACE1 and PS1 results in down-regulation of Aβ. In cirrhosis MBP is also decreased, which lead not only to functional impairment and damage of hepatic nerves, but also to reduction of NO. Reduced production of Aβ and NO upon cirrhosis may contribute to the establishment of the blood-liver barrier. Furthermore, the down-regulation of NEP and MBP in cirrhotic human liver lead to decreased clearance of Aβ delivered by the blood. Indeed, Aβ is up-regulated in the plasma of cirrhosis-affected patients.

Taken together, the results indicate that increased systemic level of Aβ during cirrhosis is explained by its impaired hepatic metabolism. The results also demonstrate that targeted Aβ construct specifically binding to HSC, alone or in combination with targeted-IFNγ and/or targeted-NO constructs are a potential therapeutic approach during advanced stages of cirrhosis.

What is claimed is:

1. A compound for use in the treatment or the prevention of a liver disease, wherein the compound is amyloid beta related protein, the amyloid beta related protein being selected from the group consisting of amyloid beta protein, an amyloid beta peptide (Aβ) derived from the amyloid beta protein, amyloid precursor protein (APP), a compound involved in the generation of an amyloid beta peptide from APP, or a compound inhibiting the degradation of the amyloid beta protein or of amyloid peptides derived therefrom.

2. The compound for use of claim 1, wherein the amyloid beta peptide derived from the amyloid beta protein is selected from the group consisting of amyloid beta 40, amyloid beta 42 and amyloid beta 38.

3. The compound for use of claim 1, wherein the compound involved in the generation of an amyloid beta peptide from APP is an enzyme selected from alpha-, beta (BACE1)-, gammasecretases, preferably presenilin.

4. The compound of claim 1, wherein the compound inhibiting the degradation of the amyloid beta protein or of amyloid peptides derived therefrom is an inhibitor of the enzyme neprilysin.

5. The compound of claim 4, wherein the inhibitor of the enzyme neprilysin is selected from sacubitril.

6. The compound of claim 1, wherein the liver disease is selected from the group consisting of liver fibrosis or cirrhosis, including primary biliary cirrhosis, nonalcoholic steatohepatitis, alcohol hepatitis, hepatocellular carcinoma and viral hepatitis.

7. A eukaryotic cell, being genetically unmodified, and naturally degrading Aβ to a lesser extent than hepatic stellate cells in the liver, for use in the treatment of a liver disease.

8. The eukaryotic cell of claim 7, wherein the eukaryotic cell is selected from astrocytes, iPS- (induced pluripotent stem cell)-derived astrocytes, and somatic cells directly reprogrammed to astrocytes, genetically modified mesenchymal stromal cells.

9. The eukaryotic cell of claim 7, wherein the liver disease is selected from the group consisting of liver fibrosis or cirrhosis, including primary biliary cirrhosis, nonalcoholic steatohepatitis, alcohol hepatitis, hepatocellular carcinoma and viral hepatitis.

10. A eukaryotic cell, being genetically modified, for use in the treatment or prevention of a liver disease, characterized in that the genetically modified eukaryotic cell has been modified to overexpress amyloid beta protein and/or amyloid beta peptides derived therefrom, APP, BACE1, and/or presenilin.

11. The eukaryotic cell of claim 9, wherein the eukaryotic cell is selected from astrocytes, iPS- (induced pluripotent stem cell)-derived astrocytes, and somatic cells directly reprogrammed to astrocytes, genetically modified mesenchymal stromal cells.

12. The eukaryotic cell of claim 10, wherein the liver disease is selected from the group consisting of liver fibrosis or cirrhosis, including primary biliary cirrhosis, nonalcoholic steatohepatitis, alcohol hepatitis, hepatocellular carcinoma and viral hepatitis.

13. A pharmaceutical composition for use in the treatment of a liver disease, especially liver fibrosis or cirrhosis, the pharmaceutical composition comprising an amyloid beta related protein, the amyloid beta related protein being selected from the group consisting of amyloid beta protein or amyloid beta peptides derived therefrom, amyloid precursor protein (APP), an enzyme involved in the generation of an amyloid beta peptide from APP, or an inhibitor of the degradation of amyloid beta protein or of amyloid beta peptides derived therefrom, and/or comprising a genetically modified eukaryotic cell that which has been modified to overexpress amyloid beta protein, APP, BACE1 and/or presenilin, together with an pharmaceutically acceptable excipient.

14. The pharmaceutical composition of claim 13, wherein the liver disease is selected from the group consisting of liver fibrosis or cirrhosis, including primary biliary cirrhosis, nonalcoholic steatohepatitis, alcohol hepatitis, hepatocellular carcinoma and viral hepatitis.

15. Method for treating or preventing a liver disease, the method comprising the step of administering to a subject in need thereof a pharmaceutically effective amount of a compound as claimed in claim 1, thereby treating or preventing the liver disease.

16. Method for treating or preventing a liver disease, the method comprising the step of administering to a subject in need thereof a pharmaceutically effective amount of a eukaryotic cell as claimed in claim 7, thereby treating or preventing the liver disease.

17. Method for treating or preventing a liver disease, the method comprising the step of administering to a subject in need thereof a pharmaceutically effective amount of a eukaryotic cell as claimed in claim 10, thereby treating or preventing the liver disease.

18. Method for treating or preventing a liver disease, the method comprising the step of administering to a subject in need thereof a pharmaceutically effective amount of a pharmaceutical composition as claimed in claim 13, thereby treating or preventing the liver disease.
